(19) United States
(12) Patent Application Publication
(10) Pub. No.: US 2022/0008642 A1
(43) Pub. Date: Jan. 13, 2022

(54) System And Apparatus For Wound Exudate Assessment

(71) Applicants: KCI Licensing, Inc., San Antonio, TX (US); Systagenix Wound Management Limited, Gatwick Airport, West Sussex (GB)

(72) Inventors: Alexander WAITE, North Yorkshire (GB); Christopher Brian LOCKE, Bournemouth (GB)

(21) Appl. No.: 17/293,190

(22) PCT Filed: Nov. 19, 2019
(86) PCT No.: PCT/US2019/062115

§ 371 Date: May 12, 2021

Related U.S. Application Data
(60) Provisional application No. 62/770,013, filed on Nov. 20, 2018.

(51) Int. Cl. A61M 1/00; A61F 13/00; A61F 13/02; A61B 10/00

(52) U.S. Cl. CPC A61M 1/95; A61B 10/0045; A61F 13/0216; A61F 13/00068

(57) ABSTRACT

In some examples, a system for treating a tissue site may include a dressing, at least one fluid sampling conduit, and at least one fluid sampling assembly. The dressing may be configured to be positioned at the tissue site. The at least one fluid sampling assembly may be configured to be in fluid communication with the tissue site through the at least one fluid sampling conduit. The at least one fluid sampling assembly may include a fluid vessel for receiving a fluid from the tissue site. The fluid may be a sampling fluid communicated directly from the tissue site and representative of the physiological condition of the tissue site. Other devices, systems, and methods are disclosed.

### RELATED APPLICATIONS

This application is a National Phase of PCT/US2019/062115, filed Nov. 19, 2019, which claims priority to U.S. Provisional Patent Application No. 62/770,013, entitled “A SYSTEM AND APPARATUS FOR WOUND EXUDATE ASSESSMENT,” filed Nov. 20, 2018, which is incorporated herein by reference for all purposes.

### TECHNICAL FIELD

The invention set forth in the appended claims relates generally to tissue treatment systems, and more particularly, but without limitation, to systems, apparatus, and methods configured to facilitate assessment or analysis of fluid exuded from a tissue site.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed. Fluid may also be managed relative to a tissue site with a suitable dressing in addition to or in lieu of negative-pressure and instillation therapies.

While the clinical benefits of fluid management relative to a tissue site are widely known, improvements to therapy systems, components, and processes may benefit healthcare providers and patients.

### BRIEF SUMMARY

New and useful systems, apparatuses, and methods for managing and monitoring fluid relative to a tissue site are set forth in the appended claims. Illustrative embodiments are also provided to enable a person skilled in the art to make and use the claimed subject matter.

In some example embodiments, a system for treating a tissue site may include a dressing, at least one fluid sampling conduit, and at least one fluid sampling assembly. The dressing may be configured to be positioned at the tissue site. The at least one fluid sampling conduit may be configured to be in direct fluid contact with the tissue site. The at least one fluid sampling assembly may be configured to be in direct fluid communication with the tissue site through the at least one fluid sampling conduit. The at least one fluid sampling assembly may include a fluid vessel. The fluid vessel may include a housing and a fluid cavity defined within the housing for receiving fluid from the tissue site.

In some example embodiments, a fluid sampling assembly may include a fluid vessel, an entry port, and a relief valve. The fluid vessel may include a housing and a fluid cavity defined within the housing. At least a portion of the housing may be moveable between a relaxed state and a compressed state. The fluid vessel may be configured to generate a sampling suction force within the fluid cavity as the housing moves from the compressed state to the relaxed state. The entry port may be positioned on the housing of the fluid vessel and configured to mate with a fluid entry valve to permit entry of a fluid into the fluid cavity by operation of the sampling suction force. The relief valve may be in fluid communication between the fluid cavity and an ambient atmosphere external to the fluid cavity. The relief valve may be configured to permit gas to exit the fluid cavity when the housing moves to the compressed state.

In some example embodiments, a fluid sampling assembly may include a fluid vessel, a fluid entry port, and a relief valve. The fluid vessel may include a housing and a fluid cavity defined within the housing. At least a portion of the fluid vessel may include a moveable component configured to vary a fluid volume of the fluid cavity between a first state and a second state. The fluid entry port may be configured to permit entry of a fluid into the fluid cavity. The relief valve may be in fluid communication between the fluid cavity and an ambient atmosphere external to the fluid cavity.

Objectives, advantages, and a preferred mode of making and using the claimed subject matter may be understood best by reference to the accompanying drawings in conjunction with the following detailed description of illustrative example embodiments.

### BRIEF DESCRIPTION OF THE DRAWINGS

FIG. 1 is a block diagram of an example embodiment of a therapy system capable of managing fluid at a tissue site and optionally providing negative-pressure treatment and instillation treatment in accordance with this disclosure;

FIG. 2 is a perspective view of example embodiments of a dressing, a fluid sampling conduit, and a fluid sampling assembly suitable for use in a system for treating a tissue site with or without negative-pressure or instillation treatment;

FIG. 3A is a plan view of an example embodiment of a bottom or tissue-facing side of the dressing of FIG. 2 that is configured to face a tissue site, illustrating an example configuration of the fluid sampling conduit relative to the dressing;

FIG. 3B is a cross-sectional view, taken at line 3B-3B in FIG. 3A, illustrating the example configuration of the fluid sampling conduit relative to the dressing;

FIG. 4A is a plan view of another example embodiment of a bottom or tissue-facing side of the dressing of FIG. 2 that is configured to face a tissue site, illustrating another example configuration of the fluid sampling conduit relative to the dressing;

FIG. 4B is a cross-sectional view, taken at line 4B-4B in FIG. 4A, illustrating another example configuration of the fluid sampling conduit relative to the dressing;

FIG. 5A is an exploded, perspective view of another example embodiment of a fluid sampling conduit;

FIG. 5B is an assembled, perspective view of the example embodiment of the fluid sampling conduit of FIG. 5A;

FIG. 6 is a bottom, perspective view of an example embodiment of a fluid sampling assembly including an example embodiment of a fluid entry valve;

FIG. 7 is a bottom, perspective view of another example embodiment of a fluid sampling assembly including another example embodiment of a fluid entry valve;

FIG. 8A is a side view of an example embodiment of a fluid sampling assembly, illustrating an example embodiment of a fluid vessel positioned in a first state or a relaxed state; and

FIG. 8B is a side view of the fluid sampling assembly of FIG. 8A, illustrating the fluid vessel positioned in a second state or a compressed state.

### DESCRIPTION OF EXAMPLE EMBODIMENTS

The following description discloses non-limiting, illustrative example embodiments with sufficient detail to enable a person skilled in the art to make and use the subject matter set forth in the appended claims. Details that are well-known or not necessary for the skilled person to make and use the claimed subject matter may be omitted.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

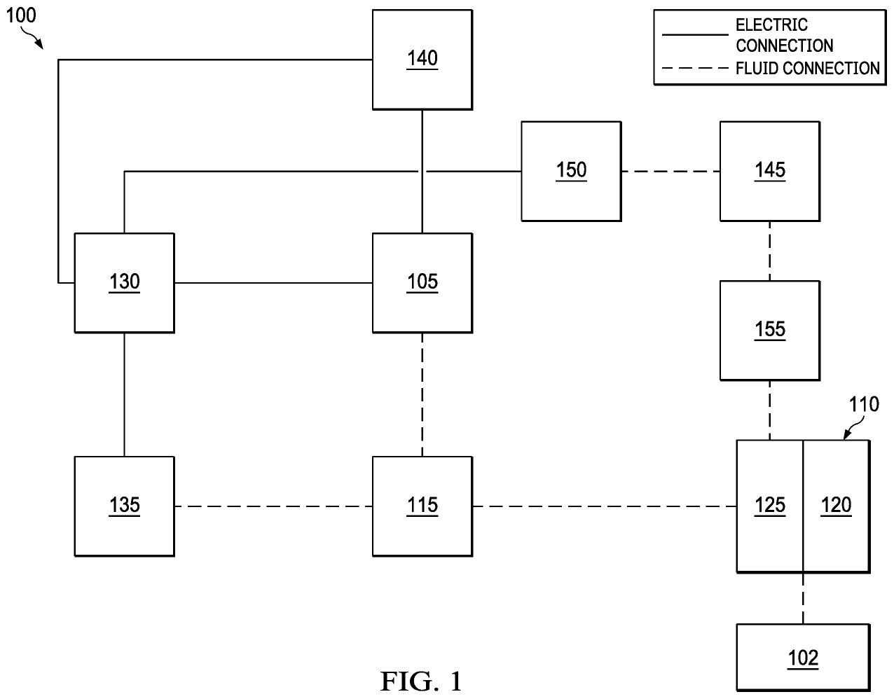

FIG. 1 is a block diagram of an example embodiment of a therapy system 100 that can optionally provide negative-pressure therapy with instillation of topical treatment solutions to a tissue site, such as a tissue site 102, in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

In some embodiments, the dressing 110 may be configured to be positioned at or in contact with the tissue site 102. Further, the negative-pressure source 105 may be referred to as a reduced-pressure source 105 and configured to be in fluid communication with the dressing 110. Further, the container 115 may be referred to as a canister 115 and configured to be in fluid communication between the dressing 110 and the reduced-pressure source 105. The canister 115 may be further configured to receive fluid from the dressing 110 and the tissue site 102. Although included as an option, in some embodiments, the reduced-pressure source 105, the canister 115, and other components may be omitted from the therapy system 100 as described herein for some therapeutic requirements or desires. Accordingly, components of the therapy system 100 are not to be deemed essential unless otherwise explicitly stated herein.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Tex.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms with or without the application of negative pressure. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity. In some embodiments, a wicking material or wicking layer may be included with or positioned proximate to the tissue interface 120 to provide or to enhance the wicking properties or the hydrophilicity of the tissue interface 120. In such an embodiment, the wicking material or wicking layer may be a non-woven or woven fibrous material, such as, for example, LIBELTEX TDL2 or LIBELTEX TL4. In some embodiments, the tissue interface 120 may include or be formed of an absorbent material, such as, without limitation, a super absorbent polymer, an absorbent foam, a hydropolymer foam, or the hydrophilic materials, foams, fibrous materials, and wicking materials described above that may also possess absorbent properties.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” may refer to a location in a fluid path relatively closer to a source of negative pressure or further away from a source of positive pressure. Conversely, the term “upstream” may refer to a location further away from a source of negative pressure or closer to a source of positive pressure.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

Further, in some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120. In some embodiments, solution may be instilled to a tissue site by applying a positive pressure from the positive-pressure source 160 to move solution from the solution source 145 to the tissue interface 120. Additionally or alternatively, the solution source 145 may be elevated to a height sufficient to allow gravity to move solution into the tissue interface 120.

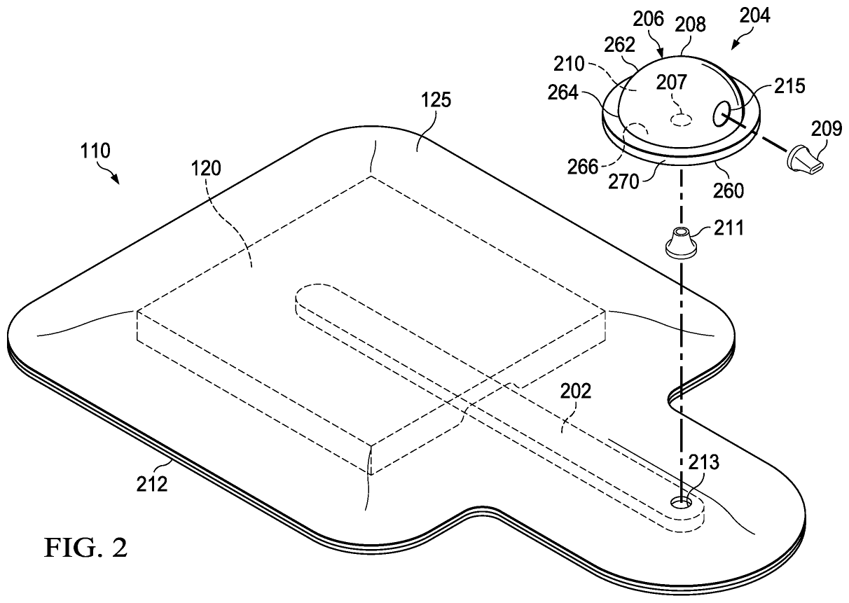

Referring to FIGS. 1-2, in some examples, the therapy system 100 may include the dressing 110, at least one fluid sampling conduit 202, and at least one fluid sampling assembly 204. The dressing 110 may be configured to be positioned at the tissue site 102. The at least one fluid sampling conduit 202 may be in fluid communication with the tissue site 102, and in some examples, may be configured to be in direct fluid contact or direct physical contact with the tissue site 102. The at least one fluid sampling assembly 204 may be configured to be in fluid communication with the tissue site 102, and in some examples, may be configured to be in direct fluid communication with the tissue site 102 through the at least one fluid sampling conduit 202. The at least one fluid sampling assembly 204 may include a fluid vessel 206. The fluid vessel 206 may include a housing 208 and a fluid cavity 210 defined within the housing 208 for receiving fluid from the tissue site 102.

In some examples, the fluid sampling assembly 204 may include an entry port 207 and a relief valve 209. The entry port 207 may be configured to permit entry of a fluid into the fluid cavity 210. For example, the entry port 207 may be positioned on the fluid vessel 206 or the housing 208, or disposed through the fluid vessel 206 or the housing 208. The entry port 207 may be configured to mate or to be fluidly coupled with a fluid entry valve 211 to permit entry of a fluid into the fluid cavity 210, for example, by operation of a sampling suction force generated by the fluid sampling assembly 204. The fluid entry valve 211 may permit entry of a fluid into the fluid cavity 210 and preclude or prevent exit of the fluid from the fluid cavity 210. In some examples, fluid may be communicated into the fluid cavity 210 by wicking or capillary forces in addition to or in lieu of the fluid sampling suction force. The entry port 207 and/or the fluid entry valve 211 may be configured to be positioned in fluid communication between the tissue site 102 and the fluid cavity 210. For example, the entry port 207 and/or the fluid entry valve 211 may be fluidly coupled to the fluid sampling conduit 202 through a sampling conduit port 213 disposed in the fluid sampling conduit 202 such that the fluid cavity 210 is in fluid communication with the tissue site 102.

The relief valve 209 may be in fluid communication between the fluid cavity 210 and an ambient atmosphere external to the fluid cavity 210. The relief valve 209 may be fluidly coupled to an exit port 215 positioned on the fluid vessel 206 or the housing 208, or disposed through the fluid vessel 206 or the housing 208. The relief valve 209 may be a one-way valve configured to permit gas to exit the fluid cavity 210 while retaining liquid, and to preclude or to prevent gas and liquid from entering the fluid cavity 210. In some examples, the relief valve 209 may be, without limitation, a duck-bill valve, check-valve, or flapper valve. In some examples, a suitable gas-permeable and liquid-impermeable filter may be incorporated in the relief valve 209 or deployed with the relief valve 209 to prevent liquid from exiting the relief valve 209.

In some examples, the fluid entry valve 211 may be coupled to the fluid sampling conduit 202 and the dressing 110. The fluid entry valve 211 may be a one-way valve configured or positioned to permit entry of a fluid into the fluid cavity 210 and to preclude exit of the fluid from the fluid cavity 210. In some embodiments, the fluid entry valve 211 may be, without limitation, a duck-bill valve, check-valve, or flapper valve.

In some examples, the fluid sampling conduit 202 and/or the fluid sampling assembly 204 may be configured to be coupled to the dressing 110 and positioned at the tissue site 102 with the dressing 110. In some examples, the fluid sampling conduit 202 and/or the fluid sampling assembly 204 may be integrally formed with the dressing 110. In other examples, the fluid sampling conduit 202 and/or the fluid sampling assembly 204 may be positioned at the tissue site prior to or during deployment of the dressing 110 at the tissue site 102.

In some examples, the at least one fluid sampling conduit 202 may include or be a plurality of fluid sampling conduits 202, and the fluid sampling assembly 204 may include or be a plurality of fluid sampling assemblies 204. At least one of the fluid sampling assemblies 204 may be positioned in fluid communication with one of the fluid sampling conduits 202. The use of multiple fluid sampling assemblies 204 may allow a caregiver to assess the physiological condition of a fluid at the tissue site 102 at different times or locations.

In some examples, the dressing 110 may include the tissue interface 120 and the tissue interface 120 may be configured to be positioned in fluid contact with the tissue site 102 or in direct physical contact with the tissue site 102. In some examples, the dressing 110 may optionally include a base layer 212, which may form part of the tissue interface 120. If included, the base layer 212 may be configured to be positioned in contact with the tissue site 102 or in direct physical contact with the tissue site 102. The base layer 212 may also be configured to be positioned between the tissue site 102 and other portions of the tissue interface 120.

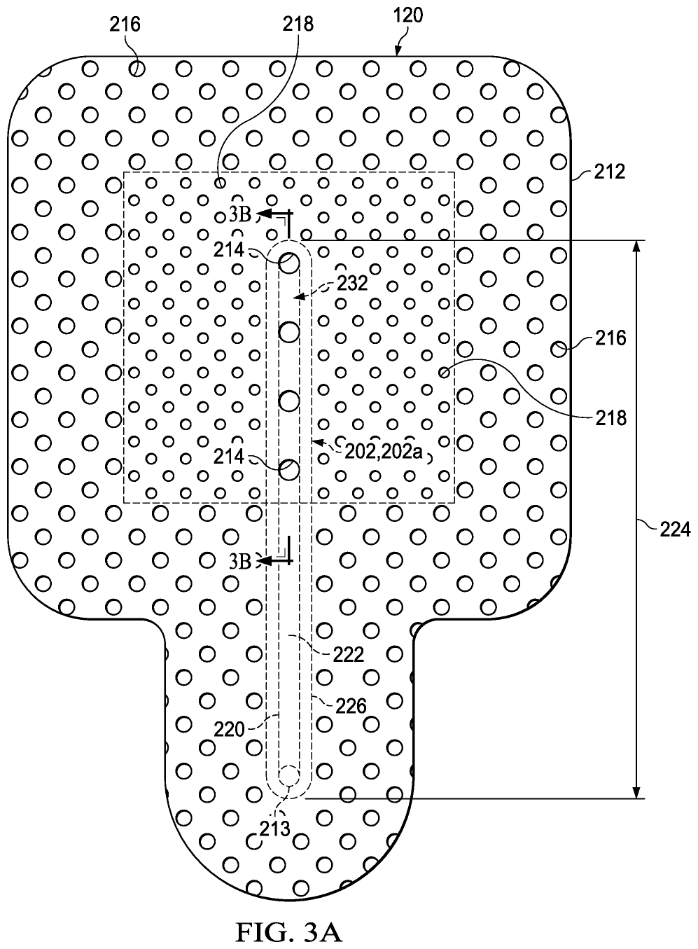

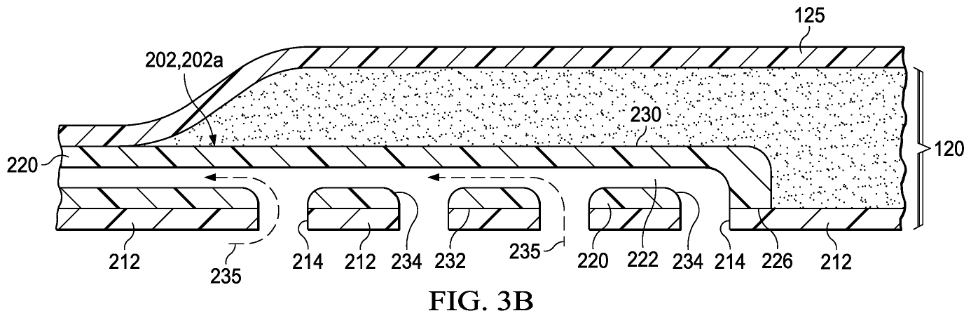

Referring to FIGS. 3A-3B, in some examples, at least one fluid sampling aperture 214 may be disposed through the tissue interface 120 or a portion of the tissue interface 120. The at least one fluid sampling conduit 202 may be configured to be in direct fluid contact with the tissue site 102 through the at least one fluid sampling aperture 214. If the base layer 212 is included, the at least one fluid sampling aperture 214 may be disposed through the base layer 212 as a portion of the tissue interface 120. In some examples, each of the at least one fluid sampling apertures 214 may be disposed entirely through opposing sides of the tissue interface 120, or the base layer 212 as an optional portion of the tissue interface 120.

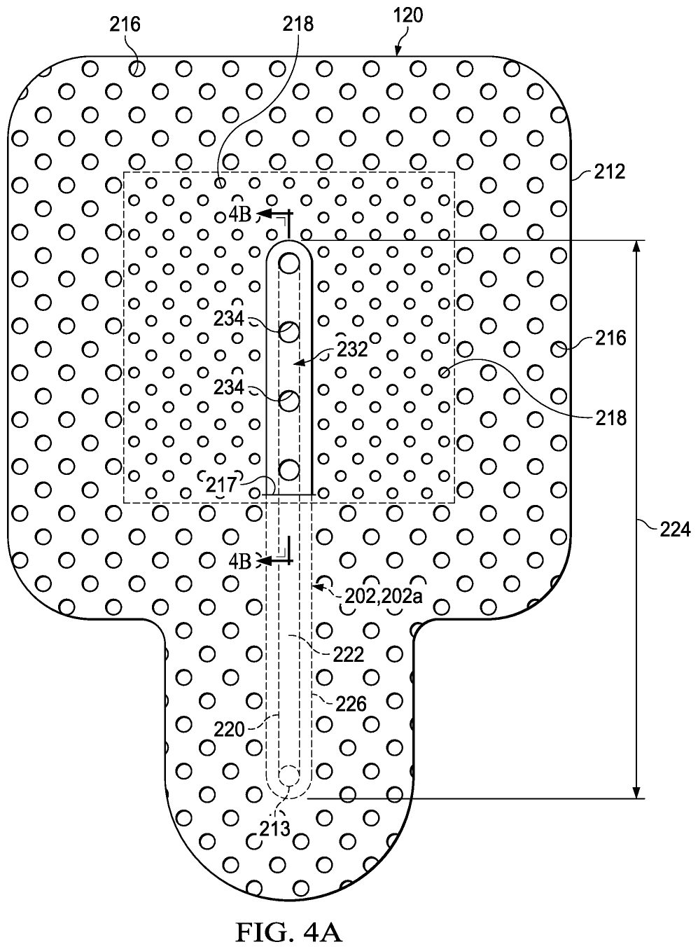

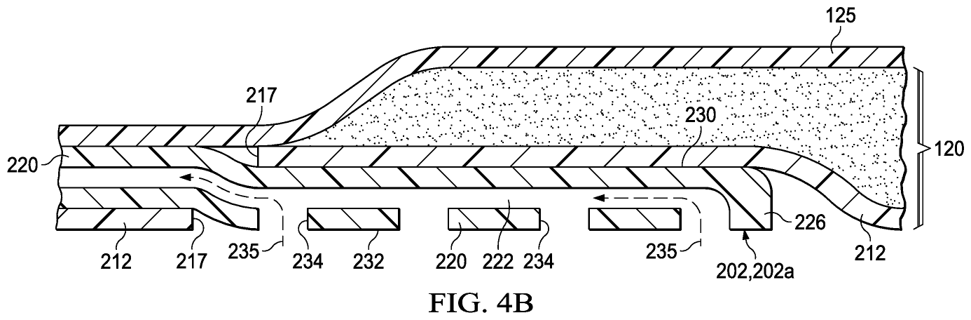

Referring to FIGS. 4A-4B, the fluid sampling conduit 202 may be configured to be positioned between the tissue site 102 and at least a portion the tissue interface 120. Further, the at least one fluid sampling conduit 202 may be configured to be in direct physical contact with the tissue site 102. For example, the at least one fluid sampling conduit 202 may be positioned directly on the tissue site 102 and/or inserted through an opening 217 in the tissue interface 120 or a portion of the tissue interface 120, such as the base layer 212. In the examples herein, the fluid sampling conduit 202 and the fluid sampling assembly 204 may be configured to receive a fluid directly from the tissue site 102 that is free of alteration, filtration, or passage through other components of the therapy system 100 or the dressing 110 capable of removing substances from the fluid, such as, without limitation, foams, meshes, gauzes, filters, fibrous materials, or other such components.

Referring to FIGS. 3A-4B, in some examples, the base layer 212 may include peripheral apertures 216 and central apertures 218. The peripheral apertures 216 are configured to be positioned around a periphery of the tissue site 102, and the central apertures 218 are configured to cover or to be positioned over the tissue site 102. The peripheral apertures 216 may permit an attachment device, such as an adhesive, to extend through the base layer 212 into contact with the periphery of the tissue site 102. The attachment device or adhesive may be positioned between the cover 125 and the base layer 212, and may be configured to adhere and to fluidly seal the cover 125 and the base layer 212 over and around the tissue site 102 when the attachment device extends through the base layer 212 to contact the periphery of the tissue site 102. The central apertures 218 may be configured to provide fluid communication between the tissue site 102 and the dressing 110 through the base layer 212. The peripheral apertures 216 and the central apertures 218 are shown in FIGS. 3A and 4A, without limitation, as being circular in shape with the peripheral apertures 216 being larger in size or diameter than the central apertures 218. However, in other examples, the peripheral apertures 216 and the central apertures 218 may have a variety of shapes and sizes to suit a particular type of therapy.

The base layer 212 may include or be formed from a soft, pliable material suitable for providing a fluid seal with the tissue site 102. For example, the base layer 212 may comprise a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins coated with an adhesive, polyurethane, polyolefin, or hydrogenated styrenic copolymers. In some examples, the base layer 212 may have a thickness between about 500 microns (μm) and about 1000 microns (μm). Further, in some examples, the base layer 212 may have a stiffness between about 5 Shore 00 and about 80 Shore 00. The base layer 212 may be comprised of hydrophobic or hydrophilic materials.

In some examples (not shown), the base layer 212 may be a hydrophobic-coated material. For example, the base layer 212 may be formed by coating a spaced material, such as, for example, woven, nonwoven, molded, or extruded mesh with a hydrophobic material. The hydrophobic material for the coating may be a soft silicone, for example. Such a configuration may permit an adhesive to extend through openings in the spaced material analogous to the peripheral apertures 216 and the central apertures 218.

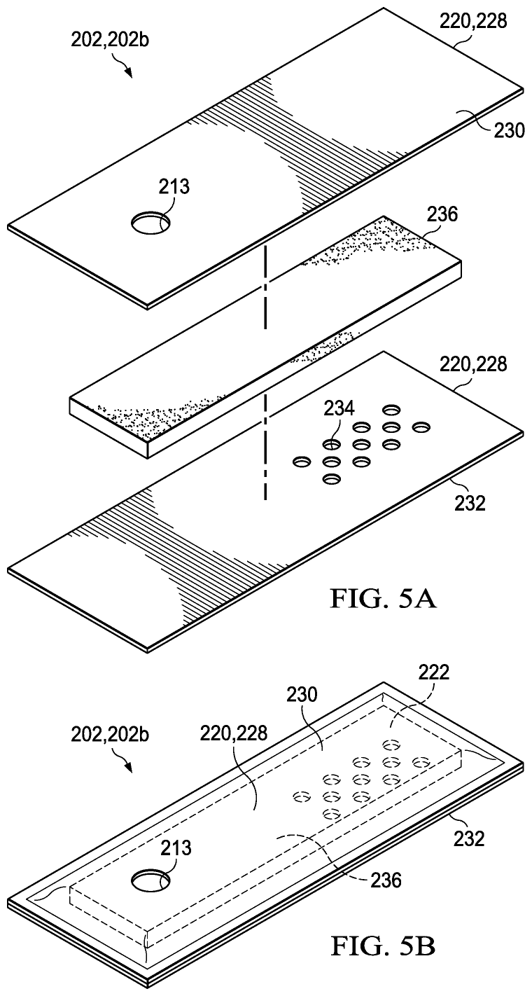

Continuing with FIGS. 3A-4B, in some examples, the at least one fluid sampling conduit 202 may include a conduit wall 220. The conduit wall 220 may define a lumen 222 extending along a length 224 of the at least one fluid sampling conduit 202. In some examples, the fluid sampling conduit 202 may be a fluid sampling conduit 202a, and the conduit wall 220 of the fluid sampling conduit 202a may be defined by a tube 226 as shown in FIGS. 3A-4B. In other examples, the fluid sampling conduit 202 may be a fluid sampling conduit 202b, and the conduit wall 220 of the fluid sampling conduit 202b may be defined by one or more layers of a film 228 as shown in FIGS. 5A-5B.

Referring to FIGS. 3A-5B, the conduit wall 220 may include a dressing-facing surface 230 configured to face the dressing 110 and a tissue-facing surface 232 configured to face the tissue site 102. In some examples, the dressing-facing surface 230 may be substantially fluid impermeable or liquid impermeable and the tissue-facing surface 232 may be fluid permeable. Further, in some examples, the conduit wall 220 may include at least one wall aperture 234 disposed through the tissue-facing surface 232. The at least one wall aperture 234 may be in fluid communication between the lumen 222 and the tissue-facing surface 232. In some examples, when positioned at the tissue site 102, the lumen 222 may be in direct fluid contact with the tissue site 102. Further, in some examples, the tissue-facing surface 232 may be configured to be positioned in direct fluid contact or direct physical contact with the tissue site 102 such that the lumen 222 is positioned direct fluid contact with the tissue site 102. Such configurations may permit a fluid, such as a sampling fluid 235, to enter the lumen 222 and the fluid cavity 210 of the fluid sampling assembly 204 that is accurately representative of the physiological condition of the tissue site 102. The sampling fluid 235 may be, for example, free of alteration, filtration, or passage through components of the dressing 110 capable of removing substances from the sampling fluid 235, such as, without limitation, foams, meshes, gauzes, filters, fibrous materials, or other such components.

Referring to FIGS. 5A-5B, in some examples, the fluid sampling conduit 202b may include a wicking material 236 disposed in the lumen 222. The wicking material 236 may include a hydrophilic gradient configured to move fluid toward the fluid sampling assembly 204. Although the wicking material 236 is shown in FIGS. 5A-5B with an example of the conduit wall 220 as the film 228, the wicking material 236 may be used with other examples, including the example of FIGS. 3A-4B where the conduit wall 220 is defined by the tube 226. The wicking material 236 may be a non-woven or woven fibrous material, such as, for example, LIBELTEX TDL2 or LIBELTEX TL4.

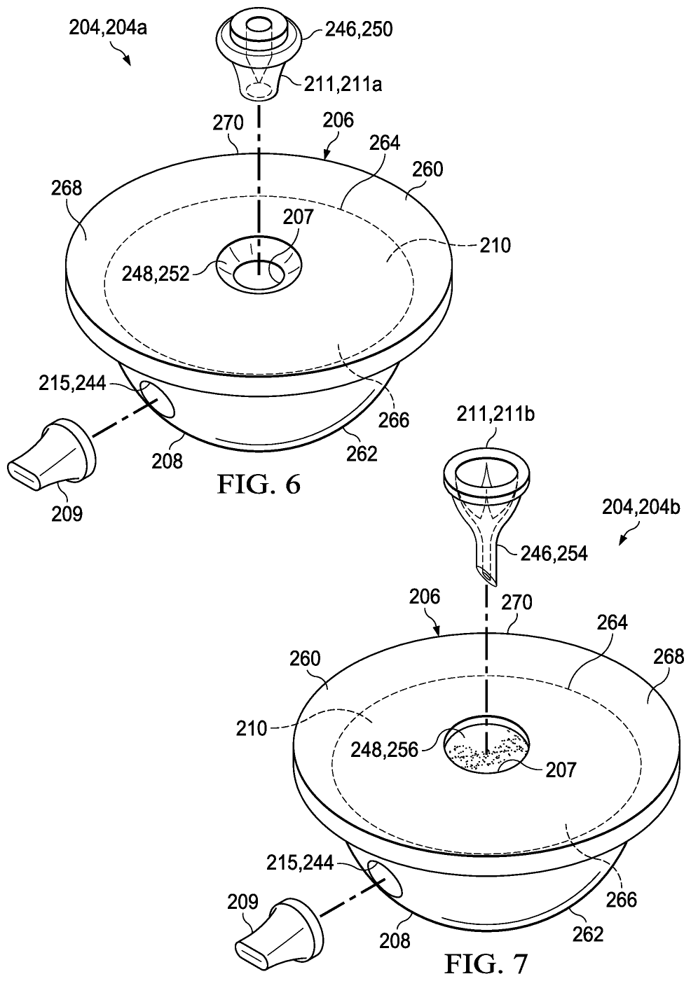

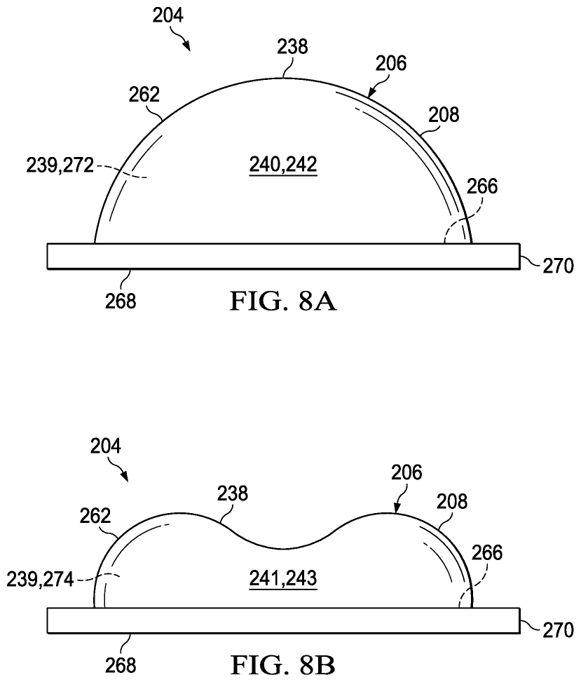

Referring to FIGS. 6-8B, in some examples, at least a portion of the fluid vessel 206 may include a moveable component 238 configured to vary a fluid volume 239 of the fluid cavity 210 between a first state 240 and a second state 241. For example, the moveable component 238 may be at least a portion of the housing 208 of the fluid sampling assembly 204 that is moveable or deformable between the first state 240, shown in FIG. 8A, and the second state 241, shown in FIG. 8B. The first state 240 may be referred to as a relaxed state 242, and the second state 241 may be referred to as a compressed state 243. In some examples, at least a portion of the housing 208 may include or be formed of a resilient material configured to return to the relaxed state 242 from the compressed state 243. For example, at least a portion of the housing 208 may include or be formed of a soft polymer, transparent polymer, or transparent film. The fluid sampling assembly 204 or the fluid vessel 206 may be configured to generate a suction force, such as a sampling suction force, within the fluid cavity 210 as the housing 208 returns to the relaxed state 242 or moves from the compressed state 243 to the relaxed state 242. In some examples, the sampling suction force may be between −20 mm Hg to −40 mm Hg and may be communicated to the tissue site 102 through the fluid sampling conduit 202.

The fluid sampling assembly 204 and the fluid vessel 206 are non-powered, and thus, the sampling suction force may be generated mechanically, for example, by potential energy that is generated by the movement of the moveable component 238 to the compressed state 243 and released as the moveable component 238 returns to the relaxed state 242. The sampling suction force may be generated by the resilience or shape of the moveable component 238 or the housing 208 without requiring expandable elements, such as foams or springs to return the moveable component 238 to the relaxed state 242. In some examples, the moveable component 238 or the housing 208 may have a convex exterior shape. Further, in some examples, a portion of the housing 208 may be deformable from the relaxed state 242 to the compressed state 243 with a compression force of 2 Newton or less.

In some examples, the fluid sampling assembly 204 may include a sampling port 244 configured to selectively provide fluid communication with the fluid cavity 210. In some examples, the relief valve 209 or the exit port 215 may provide or be utilized as the sampling port 244. In some examples, the sampling port 244 may be omitted and a fluid sample may be obtained, without limitation, by removing the fluid sampling assembly 204, cutting into the fluid sampling assembly 204, or using a syringe to pierce a portion of the fluid sampling assembly 204.

Referring to FIGS. 6-7, in some examples, the fluid entry valve 211 may include a connector 246 configured to mate with a receptor 248 carried at the entry port 207 or the housing 208 of the fluid sampling assembly 204. Referring to FIG. 6, in some examples, the fluid sampling assembly 204 may be a fluid sampling assembly 204a, and the fluid entry valve 211 may be a fluid entry valve 211a. The connector 246 of the fluid entry valve 211a may be an annular projection 250 extending circumferentially outward from and around the fluid entry valve 211a, and the receptor 248 of the fluid sampling assembly 204a may be a port detent 252 having an annular, concave shape that is positioned at the entry port 207. The annular projection 250 may have a convex shape configured to mate with the concave shape of the port detent 252.

Referring to FIG. 7, in some examples, the fluid sampling assembly 204 may be a fluid sampling assembly 204b, and the fluid entry valve 211 may be a fluid entry valve 211b. The connector 246 of the fluid entry valve 211b may include or be a tubular projection 254, and the receptor 248 of the fluid sampling assembly 204b may include or be an elastomeric membrane 256 positioned across, over, or covering the entry port 207. The tubular projection 254 may be configured to be inserted through the elastomeric membrane 256 to provide fluid communication between the fluid cavity 210 and the fluid entry valve 211b. In such non-limiting examples, the fluid entry valve 211 may remain coupled to the dressing 110 and/or the fluid sampling conduit 202 while the fluid vessel 206 of fluid sampling assembly 204 may be removed from the fluid entry valve 211 and replaced as needed or desired.

Continuing with FIGS. 6-7, in some examples, the fluid vessel 206 or the housing 208 may include a base 260 and a deformable blister 262 coupled around a periphery 264 of the base 260 and enclosing an interior surface 266 of the base 260. The fluid cavity 210 may be defined between the interior surface 266 of the base 260 and the deformable blister 262. The entry port 207 may be positioned on or through the base 260, and the base 260 may be coupled to or proximate to the dressing 110 or the tissue site 102 with an attachment device, such as an adhesive.

The base 260 may have an exterior surface 268 facing opposite from the interior surface 266 of the base 260. The base 260 may additionally include a flange 270 extending outward from and around the base 260. The exterior surface 268 of the base 260 and the flange 270 may be configured to be coupled proximate to the dressing 110 for treating the tissue site 102.

Referring to FIGS. 8A-8B, in some examples, the fluid cavity 210 may have a variable volume. For example, the fluid cavity 210 may have a relaxed volume 272 when the moveable component 238 or the housing 208 is in the relaxed state 242, and a compressed volume 274 when the moveable component 238 or the housing 208 is in the compressed state 243. The relaxed volume 272 may be greater than the compressed volume 274. In some examples, the fluid cavity 210 may have a relaxed volume 272 between about 15 milliliters to about 25 milliliters. The sampling suction force may be generated, in part, by an increase in volume of the fluid cavity 210 when the moveable component 238 or the housing 208 moves from the compressed state 243, wherein the fluid cavity 210 has the compressed volume 274, to the relaxed state 242, wherein the fluid cavity 210 has the increased relaxed volume 272.

In use, the moveable component 238 or the housing 208 may be depressed or compressed and released. When the moveable component 238 or the housing 208 is depressed or compressed, gas within the fluid cavity 210 is forced out of the relief valve 209 and precluded from re-entry. When the moveable component 238 or the housing 208 is released to permit the fluid vessel 206 to return to the relaxed state 242, the sampling suction force is generated in the form of a vacuum that is communicated through the fluid sampling conduit 202 to the tissue site 102 from which a sampling fluid is drawn into the fluid cavity 210 of the fluid sampling assembly 204. The fluid vessel 206 or the housing 208 may be removed from the dressing 110 and replaced with a new fluid vessel 206 or a new housing 208 capable drawing an additional fluid sample when desired.

A person of skill in the art will recognize numerous benefits associated with the systems, apparatus, and methods described herein. For example, the systems, apparatus, and methods are configured such that the fluid sampling assembly 204 may receive a sampling fluid from the tissue site 102 that is accurately representative of the physiological condition of the tissue site 102. The sampling fluid may be, for example, free of filtration or passage through components of the dressing 110, such as, without limitation, foams, meshes, gauzes, filters, fibrous materials, or other such components that could remove substances from the sampling fluid. The sampling fluid may be visually assessed or removed for a detailed analysis of the composition of the fluid without removal of the dressing 110 from the tissue site 102. The molecular and cellular composition of the sampling fluid can indicate wound healing progression or obstacles to wound healing progression that may be used to formulate appropriate treatment strategies for a particular tissue site or wound. Therefore, an accurate representation of the physiological condition at a tissue site may promote the development of successful treatment strategies.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for treating a tissue site, comprising:
a dressing configured to be positioned at the tissue site;
at least one fluid sampling conduit configured to be in direct fluid contact with the tissue site; and
at least one fluid sampling assembly configured to be in direct fluid communication with the tissue site through the at least one fluid sampling conduit, the at least one fluid sampling assembly comprising a fluid vessel including a housing and a fluid cavity defined within the housing for receiving fluid from the tissue site.

2. The system of claim 1, wherein the fluid sampling conduit and the fluid sampling assembly are configured to be coupled to the dressing and positioned at the tissue site with the dressing.

3. (canceled)

4. The system of claim 1, wherein the dressing comprises a tissue interface configured to be positioned in contact with the tissue site, and wherein the fluid sampling conduit is configured to be positioned between the tissue site and a portion the tissue interface.

5. The system of claim 1, wherein the dressing comprises a tissue interface and at least one fluid sampling aperture disposed through the tissue interface, wherein the at least one fluid sampling conduit is configured to be in direct fluid contact with the tissue site through the at least one fluid sampling aperture.

6. The system of claim 1, wherein the dressing comprises a tissue interface and at least one fluid sampling aperture disposed through the tissue interface, wherein each of the at least one fluid sampling apertures are disposed entirely through opposing sides of the tissue interface.

7. (canceled)

8. The system of claim 1, wherein the at least one fluid sampling conduit is configured to be in direct physical contact with the tissue site.

9. The system of claim 1, wherein the at least one fluid sampling conduit comprises a conduit wall defining a lumen extending along a length of the at least one fluid sampling conduit.

10. The system of claim 9, wherein the conduit wall is defined by a fluid impermeable film or a tube.

11. (canceled)

12. The system of claim 9, wherein the fluid sampling conduit further comprises a wicking material disposed in the lumen, wherein the wicking material includes a hydrophilic gradient configured to move fluid toward the fluid sampling assembly.

13. The system of claim 9, wherein the conduit wall comprises a dressing-facing surface configured to face the dressing and a tissue-facing surface configured to face the tissue site, and wherein the dressing-facing surface is fluid impermeable and the tissue-facing surface is fluid permeable.

14. The system of claim 13, wherein the conduit wall further comprises at least one wall aperture disposed through the tissue-facing surface, and wherein the at least one wall aperture is in fluid communication between the lumen and the tissue-facing surface.

15. The system of claim 1, wherein the at least one fluid sampling conduit comprises a plurality of fluid sampling conduits and the fluid sampling assembly comprises a plurality of fluid sampling assemblies, at least one of the fluid sampling assemblies being positioned in fluid communication with one of the fluid sampling conduits.

16. The system of claim 1, wherein at least a portion of the housing of the fluid sampling assembly is deformable from a relaxed state to a compressed state, and wherein the fluid sampling assembly is configured to generate a sampling suction force as the housing returns to the relaxed state.

17. The system of claim 1, wherein the fluid sampling assembly further comprises:
an entry port disposed through the housing of the fluid vessel and configured to mate with a fluid entry valve to permit entry of fluid into the fluid cavity; and
a relief valve in fluid communication between the fluid cavity and an ambient atmosphere external to the fluid cavity.

18. The system of claim 17, wherein the fluid entry valve is coupled to the fluid sampling conduit and the dressing, and wherein the fluid entry valve further comprises a connector configured to mate with a receptor carried at the entry port of the housing.

19. The system of claim 18, wherein the connector comprises a tubular projection and the receptor comprises an elastomeric membrane, and wherein the tubular projection is configured to be inserted through the elastomeric membrane.

20. The system of claim 1, further comprising:
a reduced-pressure source configured to be in fluid communication with the dressing; and
a canister configured to be in fluid communication between the dressing and the reduced-pressure source, wherein the canister is configured to receive fluid from the dressing and the tissue site.

21. The system of claim 20, wherein the reduced-pressure source is configured to communicate a reduced pressure to the dressing between −50 mm Hg to −300 mm Hg, and wherein the fluid sampling assembly is configured to communicate a sampling suction force to the tissue site between −20 mm Hg to −40 mm Hg.

22. A fluid sampling assembly, comprising:
a fluid vessel including a housing and a fluid cavity defined within the housing, at least a portion of the housing being moveable between a relaxed state and a compressed state, the fluid vessel being configured to generate a sampling suction force within the fluid cavity as the housing moves from the compressed state to the relaxed state;
an entry port on the housing of the fluid vessel configured to mate with a fluid entry valve to permit entry of a fluid into the fluid cavity by operation of the sampling suction force; and
a relief valve in fluid communication between the fluid cavity and an ambient atmosphere external to the fluid cavity, the relief valve configured to permit gas to exit the fluid cavity when the housing moves to the compressed state.

23.-38. (canceled)

39. A fluid sampling assembly, comprising:
a fluid vessel including a housing and a fluid cavity defined within the housing, at least a portion of the fluid vessel including a moveable component configured to vary a fluid volume of the fluid cavity between a first state and a second state;
a fluid entry port configured to permit entry of a fluid into the fluid cavity; and
a relief valve in fluid communication between the fluid cavity and an ambient atmosphere external to the fluid cavity.

40. (canceled)
